SEQUENCE LISTING (2 sequences)

SEQ ID NO: 1 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 2 - protein, 191 aa, Homo sapiens
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDANSHNDDALLKNYGLLYCFRKDMDKVETFLRIVQCRSVEGSCGF

(19) United States
(12) Patent Application Publication
(10) Pub. No.: US 2010/0022454 A1
(43) Pub. Date: Jan. 28, 2010

(54) Peptides with High Affinity for the Prolactin Receptor

(75) Inventors: Leif Norskov-Lauritsen, Tappernoje (DK); Leif Christensen, Roskilde (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaard (DK)

(21) Appl. No.: 12/443,309

(22) PCT Filed: Oct. 3, 2007
(86) PCT No.: PCT/EP07/60501

§ 371 Date: Apr. 24, 2009

(30) Foreign Application Priority Data
Oct. 3, 2006 (EP) 06121683.4

(51) Int. Cl. A61K 38/22; C07K 14/575; C12N 15/11; C12N 15/00; C12N 1/19; C12N 1/20; C07K 16/26

(52) U.S. Cl. 514/12; 530/399; 536/23.51; 435/320.1; 435/254.2; 435/252.3; 530/387.9

(57) ABSTRACT

The present invention is concerned with peptides binding to the prolactin receptor, wherein said peptides have an improved binding via binding site 1 (BS1) to the prolactin receptor. In one embodiment, said improved binding is achieved by mutation of positions 61, 71 and 73.

### FIELD OF THE INVENTION

The present invention related to variants of prolactin, which variants binds to the prolactin receptor with higher affinity as well as method for producing such variants. Such prolactin variant mutations may for instance be useful for producing prolactin antagonists for use in the treatment of for instance breast cancer.

### BACKGROUND OF THE INVENTION

Prolactin (PRL) is a cytokine with a variety of biological functions, mainly related to lactation, reproduction, osmoregulation and immunoregulation. PRL is a four-helix bundle protein of 199 residues (Somers et al., Nature 372, 478-481 (1994)). The four antiparallel α-helices of the helix bundle are numbered 1-4 as they occur from the N-terminus of the primary sequence i.e. Helix 1 (residues 15-43), Helix 2 (residues 78-103), Helix 3 (residues 111-137) and Helix 4 (residues 161-193), and PRL furthermore comprises two minor helices denoted Helix 1′ (residues 59-63) and Helix 1″ (residues 69-74), which are present in the loop connecting Helix 1 and Helix 2 (Teilum et al. J. Mol. Biol. 351, 810-823 (2005), see also FIG. 1.

PRL is a potent growth factor for mammary epithelium and PRL has been associated with the development and growth of breast tumours. Furthermore, breast cancer cell lines often over-express the PRL receptor (PRL-R). Inhibiting pituitary secretion of PRL by dopamine agonists has no effect on breast tumours and it has been established that the tumour is bypassing the effect of the dopamine agonists by its own autocrine production of PRL. Thus for treatment of breast cancer it is not sufficient to inhibit the regular pituitary PRL production, but a PRL antagonist is necessary in order to prevent binding of autocrine PRL to the PRL-R on the tumour.

PRL binds two molecules of PRL-R through two regions on PRL referred to as binding site 1 (BS1) and binding site 2 (BS2). The resulting dimerization of the receptor in a 1:2 PRL:PRL-R complex is necessary for activation of the receptor and further signal transduction. A 1:1 complex of PRL:PRL-R, formed through interactions only with the higher affinity BS1 on PRL, is inactive. Thus, variants of PRL solely able to bind via BS1 will have antagonistic properties (see for instance Clevenger et al. Endocr Rev 24, 1 (2003); Goffin et al. Endocr Rev 26, 26 (2005).

Even though there is significant homology between PRL and growth hormone, PRL does not bind to the growth hormone receptor (GH-R); however growth hormone (GH) is able to bind both GH-R and PRL-R via with different, but overlapping, sites on GH (Cunningham and Wells, Proc. Natl. Acad. Sci. USA 88, 3407 (1991)).

PRL antagonists may be created by interfering with binding of PRL-R to PRL via BS2 for instance by mutating one or more small hydrophobic residues in BS2 to for instance large polar residues (for instance G129R, see for instance Goffin et al. Endocr Rev 26, 26 (2005)) or otherwise sterically interfere with binding of PRL-R to BS2. Such a variant PRL can subsequently only bind PRL-R via BS1 and will thus have attained antagonistic properties.

Although it has been shown, that the prolactin G129R antagonists can inhibit tumour growth in vivo (Chen et al., Int. J. Oncology 20, 813-818 (2002)), it has also been stated that high level of prolactin receptor antagonists are necessary to obtain effects in vivo ( literature (Goffin et al., Endocrine Rev. 26, 400-422 (2005)). By improving pharmacokinetic parameters could lead to a compound which shows effect in vivo at a dose which is acceptable or desirable for a drug.

In order for an antagonist to compete favourably with wt PRL for BS1, the binding affinity of the antagonist to BS1 should be retained, or even improved. Residues within BS1 of the PRL antagonist could for instance be mutated with the purpose of increasing favourable interactions or creating novel interactions in the binding interface with PRL-R at BS1.

BS1 has generally been described to comprise the region bordered by Helix 1 and Helix 4 specifically involving residues Val-23, His-30, Phe-37, Lys-69, Tyr-169, His-173, Arg-176, Arg-177, His-180, Lys-181, Tyr-185, and Lys-187 (Teilum et al. J. Mol. Biol. 351, 810-823 (2005)), These results have been obtained by random mutagenesis of all PRL residues while screening for mutations that affect PRL-R binding. This is both a lengthy and potentially misleading approach due to, for instance, secondary effects of the mutations. Consequently, the creation of high affinity prolactin antagonists is problematic, since the PRL BS1 has not been precisely identified.

Mutagenesis of the prolactin molecule is for instance described in Goffin V. et al., Molecular Endocrinology 6, 1381-1392 (1992) and in Kinet S. et al, The Journal of Biological Chemistry 271, 14353-14360 (1996).

### SUMMARY OF THE INVENTION

The present invention is concerned with peptides binding to the prolactin receptor, wherein said peptides have an improved binding via binding site 1 (BS1) to the prolactin receptor.

In one embodiment, the present invention is concerned with an isolated peptide, which peptide is a variant of human prolactin, and which binds to the prolactin receptor, said variant having one or more amino acid mutations in the positions corresponding to positions 61, 71 and 73 of SEQ ID No. 1.

In one embodiment, the present invention is concerned with an isolated peptide, which peptide is a variant of human growth hormone, and which binds to the prolactin receptor, said variant having one or more amino acid mutations in the positions corresponding to positions 61, 71 and 73 of SEQ ID No. 1.

### DESCRIPTION OF THE DRAWINGS

FIG. 1. The primary sequence (using wt PRL numbering) and secondary structure of vPRL is displayed above the HX analyzed peptides (shown as horizontal bars). Peptides (residues 20-36, 40-63, 66-83, 173-185 and 189-199) identified to comprise BS1 by displaying reduced deuterium incorporation (>0.3 Da) after 1000s HX in the presence of ECD-PRL-R are coloured in grey.

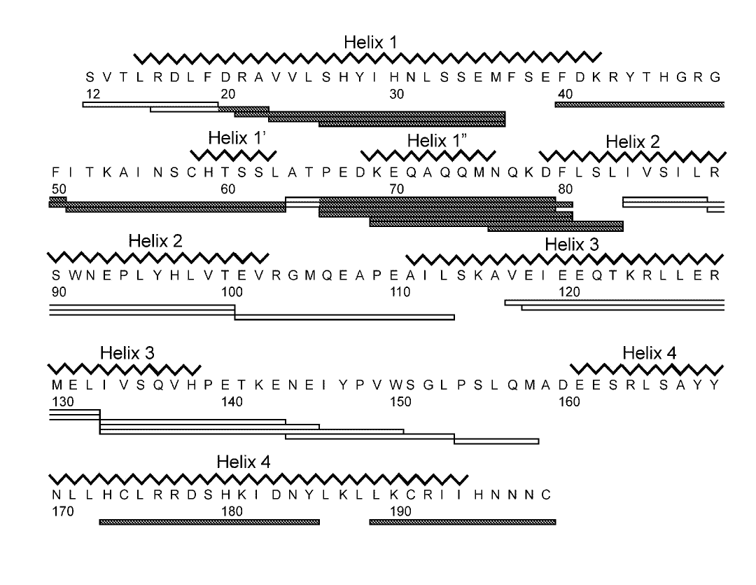

FIG. 2. Sequence alignment of prolactin and human growth hormone. Asterisk (*) denotes identical amino acids, colon (:) denotes structurally and chemically similar amino acids and point (.) denotes amino acids belonging to the same class (in casu hydrophobic or hydrophilic). The sequence listed as “prolactin” is SEQ ID No. 1, and the sequence listed as hGH is SEQ ID No. 2.

FIG. 3. Data for the binding of several PRLP binding compounds to Site 1 of PRL-R as measured in a Biocore assay.

FIG. 4. Percentage inhibition by PRL induced STAT3 phosphorylation in the presence of 0.5-10 fold of the PRL antagonist. Protein A is PRL G129R and Protein B is PRL G129R S61A. The proteins A and B were pre-mixed with PRL as follows: Column 1: Protein A 10:1. Column 2: Protein A 5:1. Column 3: Protein A 1:1. Column 4: Protein A 0.5:1. Column 5: Protein B 10:1. Column 6: Protein B 5:1. Column 7: Protein B 1:1. Column 8: Protein B 0.5:1.

FIG. 5. STAT5 tyrosin phosphorylation by western blotting.

FIG. 6. BaF/3 proliferation assay showing antagonistic effect of PRL S61A G129R.

FIG. 7. Representative graphs showing antagonistic effect in the BaF/3 assay of PRL S61A G129R and its N-terminally PEGylated analogue.

FIG. 8. Representative graphs of BaF/3 proliferation in the presence of PRL S61A G129R and its N-terminally PEGylated analogue.

### DESCRIPTION OF THE SEQUENCES

SEQ ID No. 1: Amino acid sequence for human prolactin.

SEQ ID No. 2: Amino acid sequence for human growth hormone.

### DESCRIPTION OF THE INVENTION

The present invention is concerned with peptides binding to the prolactin receptor, wherein said peptides have an improved binding via binding site 1 (BS1) to the prolactin receptor.

In one embodiment, the present invention is concerned with an isolated peptide, which peptide is a variant of human prolactin, and which binds to the prolactin receptor, said variant having one or more amino acid mutations in the positions corresponding to positions 61, 71 and 73 of SEQ ID No. 1.

In one embodiment, the present invention is concerned with an isolated peptide, which peptide is a variant of human growth hormone, and which binds to the growth hormone receptor, said variant having one or more amino acid mutations in the positions corresponding to positions 61, 71 and 73 of SEQ ID No. 1.

In one embodiment, the present invention is concerned with an isolated peptide, which peptide is a variant of human growth hormone, and which binds to the prolactin receptor, said variant having one or more amino acid mutations in the positions corresponding to positions 61, 71 and 73 of SEQ ID No. 1.

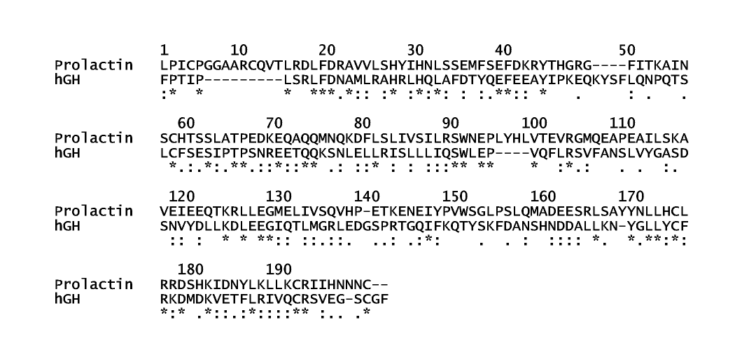

FIG. 2 shows an alignment of growth hormone to prolactin and shows which positions in growth hormone corresponds to which positions in SEQ ID No. 1.

The term “peptide” is intended to indicate a sequence of two or more amino acids joined by peptide bonds, wherein said amino acids may be natural or unnatural. The term encompasses the terms polypeptides and proteins, which may consists of two or more polypeptides held together by covalent interactions, such as for instance cysteine bridges, or non-covalent interactions. It is to be understood that the term is also intended to include peptides, which have been derivatized, for instance by the attachment of lipophilic groups, PEG or prosthetic groups. The term peptide includes any suitable peptide and may be used synonymously with the terms polypeptide and protein, unless otherwise stated or contradicted by context; provided that the reader recognize that each type of respective amino acid polymer-containing molecule may be associated with significant differences and thereby form individual embodiments of the present invention (for example, a peptide such as an antibody, which is composed of multiple polypeptide chains, is significantly different from, for example, a single chain antibody, a peptide immunoadhesin, or single chain immunogenic peptide). Therefore, the term peptide herein should generally be understood as referring to any suitable peptide of any suitable size and composition (with respect to the number of amino acids and number of associated chains in a protein molecule). Moreover, peptides in the context of the inventive methods and compositions described herein may comprise non-naturally occurring and/or non-L amino acid residues, unless otherwise stated or contradicted by context.

The term peptide, unless otherwise stated or contradicted by context,(and if discussed as individual embodiments of the term(s) polypeptide and/or protein) also encompasses derivatized peptide molecules. Briefly, in the context of the present invention, a derivative is a peptide in which one or more of the amino acid residues of the peptide have been chemically modified (for instance by alkylation, acylation, ester formation, or amide formation) or associated with one or more non-amino acid organic and/or inorganic atomic or molecular substituents (for instance a polyethylene glycol (PEG) group, a lipophilic substituent (which optionally may be linked to the amino acid sequence of the peptide by a spacer residue or group such as β-alanine, γ-aminobutyric acid (GABA), L/D-glutamic acid, succinic acid, and the like), a fluorophore, biotin, a radionuclide, etc.) and may also or alternatively comprise non-essential, non-naturally occurring, and/or non-L amino acid residues, unless otherwise stated or contradicted by context (however, it should again be recognized that such derivatives may, in and of themselves, be considered independent features of the present invention and inclusion of such molecules within the meaning of peptide is done for the sake of convenience in describing the present invention rather than to imply any sort of equivalence between naked peptides and such derivatives). Non-limiting examples of such amino acid residues include for instance 2-aminoadipic acid, 3-aminoadipic acid, β-alanine, β-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allohydroxylysine, 3-hydroxyproline, 4-hydroxyproline, isodesmosine, alloisoleucine, N-methylglycine, N-methylisoleucine, 6-N-methyllysine, N-methylvaline, norvaline, norleucine, ornithine, and statine halogenated amino acids.

In one embodiment, a peptide of the invention has an amino acid sequence having at least 80% identity to SEQ ID No. 1 including one or more amino acid mutations in the positions corresponding to positions 61, 71 and 73 of SEQ ID No. 1. In one embodiment, a peptide of the invention has an amino acid sequence having at least 85%, such as at least 90%, for instance at least 95%, such as for instance at least 99% identity to SEQ ID No. 1 including one or more amino acid mutations in the positions corresponding to positions 61, 71 and 73 of SEQ ID No. 1.

In one embodiment, a peptide of the invention has an amino acid sequence having at least 80% identity to SEQ ID No. 2 including one or more amino acid mutations in the positions corresponding to positions 61, 71 and 73 of SEQ ID No. 1. In one embodiment, a peptide of the invention has an amino acid sequence having at least 85%, such as at least 90%, for instance at least 95%, such as for instance at least 99% identity to SEQ ID No. 2 including one or more amino acid mutations in the positions corresponding to positions 61, 71 and 73 of SEQ ID No. 1.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, a peptide of the invention has an amino acid sequence, which sequence is at least 80% similar to SEQ ID No. 1 including one or more amino acid mutations in the positions corresponding to positions 61, 71 and 73 of SEQ ID No. 1. In one embodiment, a peptide of the invention has an amino acid sequence, which sequence is at least 85%, such as at least 90%, for instance at least 95%, such as for instance at least 99% similar to SEQ ID No. 1 including one or more amino acid mutations in the positions corresponding to positions 61, 71 and 73 of SEQ ID No. 1.

In one embodiment, a peptide of the invention has an amino acid sequence, which sequence is at least 80% similar to SEQ ID No. 2 including one or more amino acid mutations in the positions corresponding to positions 61, 71 and 73 of SEQ ID No. 1. In one embodiment, a peptide of the invention has an amino acid sequence, which sequence is at least 85%, such as at least 90%, for instance at least 95%, such as for instance at least 99% similar to SEQ ID No. 2 including one or more amino acid mutations in the positions corresponding to positions 61, 71 and 73 of SEQ ID No. 1.

The term “similarity” is a concept related to identity, but in contrast to “identity”, refers to a sequence relationship that includes both identical matches and conservative substitution matches. If two polypeptide sequences have, for example, (fraction (10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction (15/20))). Therefore, in cases where there are conservative substitutions, the degree of similarity between two polypeptides will be higher than the percent identity between those two polypeptides.

Conservative modifications a peptide comprising an amino acid sequence of SEQ ID No. 1 (and the corresponding modifications to the encoding nucleic acids) will produce peptides having functional and chemical characteristics similar to those of a peptide comprising an amino acid sequence of SEQ ID No. 1. In contrast, substantial modifications in the functional and/or chemical characteristics of peptides according to the invention as compared to a peptide comprising an amino acid sequence of SEQ ID No. 1 may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl. 643, 55-67 (1998); Sasaki et al., Adv. Biophys. 35, 1-24 (1998), which discuss alanine scanning mutagenesis).

Desired amino acid substitutions (whether conservative or non-conservative) may be determined by those skilled in the art at the time such substitutions are desired. For example, amino acid substitutions can be used to identify important residues of the peptides according to the invention, or to increase or decrease the affinity of the peptides described herein for the receptor in addition to the already described mutations.

Naturally occurring residues may be divided into classes based on common side chain properties:
1) hydrophobic: norleucine, Met, Ala, Val, Leu, Iie;
2) neutral hydrophilic: Cys, Ser. Thr, Asn, Gln;
3) acidic: Asp, Glu;
4) basic: His, Lys, Arg;
5) residues that influence chain orientation: Gly, Pro; and
6) aromatic: Trp, Tyr, Phe.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157, 105-131 (1982). It is known that certain amino acids may be substituted for other amino acids having a similar hydropathic index or score and still retain a similar biological activity. In making changes based upon the hydropathic index, the substitution of amino acids whose hydropathic indices are within .±2 is preferred, those that are within ±1 are particularly preferred, and those within ±0.5 are even more particularly preferred.

It is also understood in the art that the substitution of like amino acids may be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (′3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred. One may also identify epitopes from primary amino acid sequences on the basis of hydrophilicity. These regions are also referred to as “epitopic core regions”.

Peptides of the present invention may also include non-naturally occurring amino acids.

In one embodiment, a peptide according to the invention has an amino acid mutation in a position in close proximity to the position corresponding to position 73 SEQ ID No. 1.

In one embodiment, the peptide according to the invention has an increased affinity to the prolactin receptor as compared to human prolactin. In one embodiment, the affinity to the prolactin receptor is determined according to Assay (I) as described herein.

In one embodiment, the peptide according to the invention has an increased binding to the prolactin receptor through binding site 1 as compared to human prolactin. This may be taking the peptide according to the invention, introducing a mutation abolishing or significantly reducing binding to binding site 2, such as for instance G129R, measuring the binding of this molecule to the prolactin receptor and comparing this binding with binding of an otherwise similar molecule not carrying a mutation according to the invention. It is within the knowledge of a person skilled in the art to design similar methods for determining differences in binding to BS1 and/or BS2 between the wildtype prolactin (SEQ ID No. 1) and peptides according to the present invention.

In one embodiment, the binding of said peptide for the prolactin receptor has a dissociation constant (K_{d}) at least three times less than that of wildtype human PRL binding to the prolactin receptor.

In one embodiment, a peptide according to the invention, in addition to having an improved binding to BS 1 in accordance with the present invention, is also an antagonist of the prolactin receptor. In one embodiment, said antagonism is determined using Assay (II) as described herein. In one embodiment, said antagonism is achieved by introducing one or more mutations into BS2 to prevent or reduce interaction of BS2 with PRL-R. In one embodiment, at least one or more of said antagonistic mutations are selected from mutations in the amino acid residues corresponding to Gly-129 and Ser-179. In one embodiment, at least one or more of said antagonistic mutations are selected from mutations corresponding to G129R and S179D. In one embodiment, at least one or more of said antagonistic mutations are selected from a mutation corresponding to G129R. In one embodiment, amino acid residues corresponding to positions 1 to 9 in PRL have been deleted. In one embodiment, amino acid residues corresponding to positions 1 to 11 in PRL have been deleted. In one embodiment, amino acid residues corresponding to positions 1 to 14 in PRL have been deleted. In one embodiment, said antagonism is achieved by derivatizing PRL for instance by conjugation to a PEG molecule or other bulky groups, the introduction of which impairs antagonistic properties to the PRL. In one such embodiment, such a group, for instance a PEG molecule, is added in the N-terminal of PRL.

In one embodiment, the peptide according to the invention comprises one or more amino acid mutations, which stabilizes the structure of the prolactin molecule. In one embodiment, said peptide further comprises one or more amino acid mutations, which stabilizes the secondary structure of the prolactin molecule (the stabilization may in one embodiment be determined by use of HX-MS technology). In one embodiment, one or more of said amino acid mutation(s) stabilizes the 4-helix bundle structure in prolactin. In one embodiment, one or more of said amino acid mutation(s) improves the helix capping in helix 1, helix 2, helix 3 and/or helix 4 of PRL. In one embodiment, one or more of said amino acid mutation(s) introduces salt bridges in helical segments exposed to solvent. In one embodiment, two or more of said amino acid mutation(s) introduces non-native disulfide bonds into prolactin. In one embodiment, one or more of said amino acid mutation(s) is a substitution of a solvent exposed hydrophobic residue with a polar residue. In one embodiment, one or more of said amino acid mutation(s) improves the packing interactions at the hydrophobic core of the 4-helix bundle structure.

The HX-MS technology exploits that hydrogen exchange (HX) of a protein can readily be followed by mass spectrometry (MS). By replacing the aqueous solvent containing hydrogen with aqueous solvent containing deuterium, incorporation of a deuterium atom at a given site in a protein will give rise to an increase in mass of 1 Da. This mass increase can be monitored as a function of time by mass spectrometry in quenched samples of the exchange reaction.

One use of HX-MS is to probe for sites involved in molecular interactions by identifying regions of reduced hydrogen exchange upon protein-protein complex formation. Usually, binding interfaces will be revealed by marked reductions in hydrogen exchange due to steric exclusion of solvent.

Protein-protein complex formation may be detected by HX-MS simply by measuring the total amount of deuterium incorporated in either protein members in the presence and absence of the respective binding partner as a function of time. Furthermore, the deuterium labels can be sub-localized to specific regions of either protein by proteolytic fragmentation of the deuterated protein sample into short peptides and analysis of the deuteron content of each peptide. Peptides that display altered deuterium levels in the presence of binding partner either constitute or are structurally linked to the binding interface (for a recent review on the HX-MS technology see Wales and Engen, Mass Spectrom. Rev. 25, 158 (2006)). A relevant example of application of the HX-MS technology may be found in Horn et al., Biochemistry 45, 8488-8498 (2006).

Peptides and pharmaceutical compositions according to the present invention may be used in the treatment of diseases treatable by administration of prolactin antagonists, such as breast cancer.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active peptides to prevent the onset of the symptoms or complications. The patient to be treated is preferably a mammal, in particular a human being, but it may also include animals, such as dogs, cats, cows, sheep and pigs. It is to be understood, that therapeutic and prophylactic (preventive) regimes represent separate aspects of the present invention.

A “therapeutically effective amount” of a peptide as used herein means an amount sufficient to cure, alleviate or partially arrest the clinical manifestations of a given disease and its complications. An amount adequate to accomplish this is defined as “therapeutically effective amount”. Effective amounts for each purpose will depend on the type and severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a peptide of interest. The construct may optionally contain other nucleic acid segments.

A nucleic acid construct of the invention may suitably be of genomic or cDNA origin, for instance obtained by preparing a genomic or cDNA library and screening for DNA sequences coding for all or part of the peptide by hybridization using synthetic oligonucleotide probes in accordance with standard techniques (cf. J. Sambrook et al, 1989, Molecular Cloning, A Laboratory Manual, 2d edition, Cold Spring Harbor, N.Y.) and by introducing the relevant mutations as it is known in the art.

A nucleic acid construct of the invention may also be prepared synthetically by established standard methods, e.g. the phosphoamidite method described by Beaucage and Caruthers, Tetrahedron Letters 22, 1859-1869 (1981), or the method described by Matthes et al., EMBO Journal 3, 801-805 (1984). According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239, 487-491 (1988).

In one embodiment, the nucleic acid construct of the invention is a DNA construct which term will be used exclusively in the following for convenience. The statements in the following may also read on other nucleic acid constructs of the invention with appropriate adaptions as it will be clear for a person skilled in the art.

In one embodiment, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector may be an expression vector in which the DNA sequence encoding the peptide of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the peptide.

The promoter may be any DNA sequence which shows transcriptional activity in the host cell of choice and may be derived from genes encoding proteins either homologous or heterologous to the host cell.

Examples of suitable promoters for use in yeast host cells include promoters from yeast glycolytic genes (Hitzeman et al., J. Biol. Chem. 255, 12073-12080 (1980); Alber and Kawasaki, J. Mol. Appl. Gen. 1, 419-434 (1982)) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304, 652-654 (1983)) promoters.

Examples of suitable promoters for use in filamentous fungus host cells are, for instance, the ADH3 promoter (McKnight et al., The EMBO J. 4, 2093-2099 (1985)) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. In one embodiment, the promoter of a vector according to the invention is selected from the TAKA-amylase or the gluA promoters.

Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gen, or the Bacillus pumilus xylosidase gene, or by the phage Lambda P_{R} or P_{L} promoters or the E. coli lac, trp or tac promoters.

The DNA sequence encoding the peptide of the invention may also, if necessary, be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector of the invention may further comprise a DNA sequence enabling the vector to replicate in the host cell in question.

When the host cell is a yeast cell, suitable sequences enabling the vector to replicate are the yeast plasmid 2μ replication genes REP 1-3 and origin of replication.

When the host cell is a bacterial cell, sequences enabling the vector to replicate are DNA polymerase III complex encoding genes and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 125-130 (1985)), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD and sC.

To direct a peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

For secretion from yeast cells, the secretory signal sequence may encode any signal peptide which ensures efficient direction of the expressed peptide into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 643-646 (1981)), a modified carboxypeptidase signal peptide (cf. L. A. Valls et al., Cell 48, 887-897 (1987)), the yeast BAR1 signal peptide (cf. WO 87/02670), or the yeast aspartic protease 3 (YAP3) signal peptide (cf. M. Egel-Mitani et al., Yeast 6, 127-137 (1990)).

For efficient secretion in yeast, a sequence encoding a leader peptide may also be inserted downstream of the signal sequence and uptream of the DNA sequence encoding the peptide. The function of the leader peptide is to allow the expressed peptide to be directed from the endoplasmic reticulum to the Golgi apparatus and further to a secretory vesicle for secretion into the culture medium (i.e. exportation of the peptide across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide may be derived from a gene encoding A. oryzae TAKA amylase, A. niger neutral α-amylase, A. niger acid-stable amylase, or A. niger glucoamylase.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., op.cit.).

The host cell into which the DNA construct or the recombinant vector of the invention is introduced may be any cell which is capable of producing the present peptide and includes bacteria, yeast, fungi and higher eukaryotic cells.

Examples of bacterial host cells which, on cultivation, are capable of producing the peptide of the invention are grampositive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gramnegative bacteria such as Echerichia coli. The transformation of the bacteria may be effected by protoplast transformation or by using competent cells in a manner known per se (cf. Sambrook et al., supra). Other suitable hosts include S. mobaraense, S. lividans, and C. glutamicum (Appl. Microbiol. Biotechnol. 64, 447-454 (2004)).

When expressing the peptide in bacteria such as E. coli, the peptide may be retained in the cytoplasm, typically as insoluble granules (known as inclusion bodies), or may be directed to the periplasmic space by a bacterial secretion sequence. In the former case, the cells are lysed and the granules are recovered and denatured after which the peptide is refolded by diluting the denaturing agent. In the latter case, the peptide may be recovered from the periplasmic space by disrupting the cells, e.g. by sonication or osmotic shock, to release the contents of the periplasmic space and recovering the peptide.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous proteins therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. An example of a vector for use in yeast is the POT1 vector disclosed in U.S. Pat. No. 4,931,373. The DNA sequence encoding the peptide of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 3459-3465 (1986); U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardieret al. Gene 78, 147-156 (1989).

When a filamentous fungus is used as the host cell, it may be transformed with the DNA construct of the invention, conveniently by integrating the DNA construct in the host chromosome to obtain a recombinant host cell. This will make it more likely that the DNA sequence will be stably maintained in the cell. Integration of the DNA constructs into the host chromosome may be performed according to conventional methods, e.g. by homologous or heterologous recombination.

The transformed or transfected host cell described above is then cultured in a suitable nutrient medium under conditions permitting the expression of the present peptide, after which the resulting peptide is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of peptide in question.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### Pharmaceutical Compositions

The present invention provides a pharmaceutical formulation comprising a peptide of the present invention which is present in a concentration from 10⁻¹⁵ mg/ml to 200 mg/ml, such as 10⁻¹⁰ mg/ml-5 mg/ml, and wherein said formulation has a pH from 2.0 to 10.0. Optionally, said formulation may comprise one or more further cancer agents as described above. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In one embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In one embodiment the pharmaceutical formulation is a freeze-dried formulation, whereto the physician or the patient adds solvents and/or diluents prior to use.

In one embodiment the pharmaceutical formulation is a dried formulation (e.g. freeze-dried or spray-dried) ready for use without any prior dissolution.

In one embodiment the invention relates to a pharmaceutical formulation comprising an aqueous solution of a peptide of the present invention, and a buffer, wherein said OGP protein is present in a concentration from 0.1-100 mg/ml, and wherein said formulation has a pH from about 2.0 to about 10.0.

In one embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In one embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In one embodiment of the invention the formulation further comprises a pharmaceutically acceptable preservative. In one embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In one embodiment of the invention the preservative is present in a concentration from 0.1 mg/ml to 20 mg/ml. In one embodiment of the invention the preservative is present in a concentration from 0.1 mg/ml to 5 mg/ml. In one embodiment of the invention the preservative is present in a concentration from 5 mg/ml to 10 mg/ml. In one embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In one embodiment of the invention the formulation further comprises an isotonic agent. In one embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In one embodiment of the invention the isotonic agent is present in a concentration from 1 mg/ml to 50 mg/ml. In one embodiment of the invention the isotonic agent is present in a concentration from 1 mg/ml to 7 mg/ml. In one embodiment of the invention the isotonic agent is present in a concentration from 8 mg/ml to 24 mg/ml. In one embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In one embodiment of the invention the formulation further comprises a chelating agent. In one embodiment of the invention the chelating agent is selected from salts of ethylenediaminetetraacetic acid (EDTA), citric acid, and aspartic acid, and mixtures thereof. In one embodiment of the invention the chelating agent is present in a concentration from 0.1 mg/ml to 5 mg/ml. In one embodiment of the invention the chelating agent is present in a concentration from 0.1 mg/ml to 2 mg/ml. In one embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In one embodiment of the invention the formulation further comprises a stabilizer. The use of a stabilizer in pharmaceutical compositions is well-known to the skilled person. For convenience reference is made to Remington: The Science and Practice of Pharmacy, 20^{th} edition, 2000.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or mixtures thereof) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In one embodiment of the invention the amino acids or amino acid analogues are used in a concentration, which is sufficient to prevent or delay aggregation of the protein.

In one embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or mixtures thereof) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In one embodiment of the invention the formulation further comprises a stabilizer selected from the group of high molecular weight polymers or low molecular compounds. In one embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In one embodiment of the invention the formulation further comprises a surfactant. In one embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a peptide of the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the peptide of the present invention, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of a peptide of the present invention, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the peptide of the present invention in the form of a nasal or pulmonal spray. As a still further option, the pharmaceutical compositions containing the peptide of the present invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the peptide of the present invention is stable for more than 6 weeks of usage and for more than 3 years of storage.

In one embodiment of the invention the pharmaceutical formulation comprising the peptide of the present invention is stable for more than 4 weeks of usage and for more than 3 years of storage.

In one embodiment of the invention the pharmaceutical formulation comprising the peptide of the present invention is stable for more than 4 weeks of usage and for more than two years of storage.

In one embodiment of the invention the pharmaceutical formulation comprising the peptide of the present invention is stable for more than 2 weeks of usage and for more than two years of storage.

The following is a non-limiting list of exemplary embodiments of the invention.

### Embodiment 1

An isolated peptide, which peptide is a variant of human prolactin, and which binds to the prolactin receptor, said variant having one or more amino acid mutations in the positions corresponding to positions 61, 71 and 73 of SEQ ID No. 1.

### Embodiment 2

An isolated peptide, which peptide is a variant of human prolactin, and which binds to the prolactin receptor, and which peptide comprises the amino acid sequence of SEQ ID No. 1 having one or more amino acid mutations in the positions corresponding to positions 61, 71 and 73 of SEQ ID No. 1.

### Embodiment 3

An isolated peptide according to embodiment 1 or embodiment 2 having an amino acid mutation in the position corresponding to position 61 of SEQ ID No. 1.

### Embodiment 4

An isolated peptide according to embodiment 3, wherein the amino acid residue in the position corresponding to position 61 of SEQ ID No. 1 has been substituted with an alanine.

### Embodiment 5

An isolated peptide according to any of embodiments 1 to 4 having an amino acid mutation in the position corresponding to position 71 of SEQ ID No. 1.

### Embodiment 6

An isolated peptide according to embodiment 5, wherein the amino acid residue in the position corresponding to position 71 of SEQ ID No. 1 has been substituted with an alanine.

### Embodiment 7

An isolated peptide according to any of embodiments 1 to 6 having an amino acid mutation in the position corresponding to position 73 of SEQ ID No. 1.

### Embodiment 8

An isolated peptide according to embodiment 7, wherein the amino acid residue in the position corresponding to position 73 of SEQ ID No. 1 has been substituted with an leucine.

### Embodiment 9

An isolated peptide according to embodiment 7, wherein the amino acid residue in the position corresponding to position 73 of SEQ ID No. 1 has been substituted with an alanine.

### Embodiment 10

An isolated peptide according to any of embodiments 1 to 9, wherein said peptide has an increased affinity to the prolactin receptor as compared to human prolactin.

### Embodiment 11

An isolated peptide according to embodiment 10, wherein the affinity to the prolactin receptor is determined according to Assay (I) as described herein.

### Embodiment 12

An isolated peptide according to any of embodiments 1 to 11, wherein the peptide has an increased binding to the prolactin receptor through binding site 1 as compared to human prolactin.

### Embodiment 13

An isolated peptide according to any of embodiments 1 to 12, wherein the binding of said peptide for the prolactin receptor has a dissociation constant (K_{d}) at least three times less than that of wildtype human PRL binding to the prolactin receptor.

### Embodiment 14

An isolated peptide according to any of embodiments 1 to 13, wherein said peptide is capable of binding to the human growth hormone receptor.

### Embodiment 15

An isolated peptide according to embodiment 14, wherein the binding to the human growth hormone receptor is determined by use of an assay as described as Assay (I) herein.

### Embodiment 16

An isolated peptide according to any of embodiments 1 to 15, which is an antagonist of the prolactin receptor.

### Embodiment 17

An isolated peptide according to embodiment 16, wherein said antagonism is determined using Assay (II) as described herein.

### Embodiment 18

An isolated peptide according to embodiment 16 or embodiment 17, wherein said antagonism is achieved by introducing one or more mutations into BS2 to prevent or reduce interaction of BS2 with PRL-R.

### Embodiment 19

An isolated peptide according to any of embodiments 16 to 18, wherein at least one or more of said antagonistic mutations are selected from mutations in the amino acid residues corresponding to Gly-129 and Ser-179.

### Embodiment 20

An isolated peptide according to embodiment 19, wherein at least one or more of said antagonistic mutations are selected from mutations corresponding to G129R and S179D.

### Embodiment 21

An isolated peptide according to embodiment 20, wherein at least one or more of said antagonistic mutations are selected from a mutation corresponding to G129R.

### Embodiment 22

An isolated peptide according to embodiment 21, wherein the amino acid residues corresponding to positions 1 to 9 in PRL have been deleted.

### Embodiment 23

An isolated peptide according to embodiment 22, wherein the amino acid residues corresponding to positions 1 to 14 in PRL have been deleted.

### Embodiment 24

An isolated peptide according to any of embodiments 1 to 15, which is an agonist of the prolactin receptor.

### Embodiment 25

An isolated peptide according to embodiment 24, wherein said peptide binds binding site 2.

### Embodiment 26

An isolated peptide according to any of embodiments 1 to 25, wherein said peptide comprises one or more amino acid mutations, which stabilizes the structure of the prolactin molecule.

### Embodiment 27

An isolated peptide according to embodiment 26, wherein said variant comprises one or more amino acid mutations, which stabilizes the secondary structure of the prolactin molecule.

### Embodiment 28

An isolated peptide according to embodiment 26 or embodiment 27, wherein the stabilization of PRL is determined by use of HX-MS technology.

### Embodiment 29

An isolated peptide according to any of embodiments 26 to 28, wherein one or more of said amino acid mutation(s) stabilizes the 4-helix bundle structure in prolactin.

### Embodiment 30

An isolated peptide according to any of embodiments 26 to 29, wherein one or more of said amino acid mutation(s) improves the helix capping in helix 1, helix 2, helix 3 and/or helix 4 of PRL.

### Embodiment 31

An isolated peptide according to any of embodiments 26 to 30, wherein one or more of said amino acid mutation(s) introduces salt bridges in helical segments exposed to solvent.

### Embodiment 32

An isolated peptide according to any of embodiments 26 to 31, wherein two or more of said amino acid mutation(s) introduces non-native disulfide bonds into prolactin.

### Embodiment 33

An isolated peptide according to any of embodiments 26 to 32, wherein one or more of said amino acid mutation(s) is a substitution of a solvent exposed hydrophobic residue with a polar residue.

### Embodiment 34

An isolated peptide according to any of embodiments 26 to 33, wherein one or more of said amino acid mutation(s) improves the packing interactions at the hydrophobic core of the 4-helix bundle structure.

### Embodiment 35

An isolated peptide, which peptide is a variant of human growth hormone, and which binds to the growth hormone receptor, said variant having one or more amino acid mutations in the positions corresponding to positions 61, 71 and 73 of SEQ ID No. 1.

### Embodiment 36

An isolated peptide, which peptide is a variant of human growth hormone, and which binds to the prolactin receptor, and which peptide comprises the amino acid sequence of SEQ ID No. 2 having one or more amino acid mutations in the positions corresponding to positions 61, 71 and 73 of SEQ ID No. 1.

### Embodiment 37

An isolated peptide according to embodiment 35 or embodiment 36 having an amino acid mutation in the position corresponding to position 61 of SEQ ID No. 1.

### Embodiment 38

An isolated peptide according to embodiment 37, wherein the amino acid residue in the position corresponding to position 61 of SEQ ID No. 1 has been substituted with an alanine.

### Embodiment 39

An isolated peptide according to any of embodiments 35 to 38 having an amino acid mutation in the position corresponding to position 71 of SEQ ID No. 1.

### Embodiment 40

An isolated peptide according to embodiment 39, wherein the amino acid residue in the position corresponding to position 71 of SEQ ID No. 1 has been substituted with an alanine.

### Embodiment 41

An isolated peptide according to any of embodiments 35 to 40 having an amino acid mutation in the position corresponding to position 73 of SEQ ID No. 1.

### Embodiment 42

An isolated peptide according to embodiment 41, wherein the amino acid residue in the position corresponding to position 73 of SEQ ID No. 1 has been substituted with a leucine.

### Embodiment 43

An isolated peptide according to embodiment 41, wherein the amino acid residue in the position corresponding to position 73 of SEQ ID No. 1 has been substituted with an alanine.

### Embodiment 44

An isolated peptide according to any of embodiments 35 to 43, wherein said peptide is also mutated in one or more positions corresponding to amino acid residues 20 to 36 and/or 40 to 63 and/or 173 to 185 of SEQ ID No. 1.

### Embodiment 45

An isolated peptide according to any of embodiments 35 to 44, wherein said peptide has an increased affinity to the prolactin receptor as compared to human growth hormone.

### Embodiment 46

An isolated peptide according to embodiment 45, wherein the affinity to the prolactin receptor is determined according to Assay (I) as described herein.

### Embodiment 47

An isolated peptide according to any of embodiments 1 to 46, wherein the peptide has an increased binding to the prolactin receptor through binding site 1 as compared to human prolactin.

### Embodiment 48

An isolated peptide according to any of embodiments 35 to 47, wherein the binding of said peptide for the prolactin receptor has a dissociation constant (K_{d}) at least three times less than that of wildtype human growth hormone binding to the prolactin receptor.

### Embodiment 49

An isolated peptide according to any of embodiments 35 to 48, wherein said peptide has an increased affinity to the growth hormone receptor as compared to human growth hormone.

### Embodiment 50

An isolated peptide according to embodiment 49, wherein the affinity to the growth hormone is determined according to Assay (I) as described herein.

### Embodiment 51

An isolated peptide according to any of embodiments 35 to 50, wherein the binding of said peptide for the growth hormone receptor has a dissociation constant (K_{d}) at least three times less than that of wildtype human growth hormone binding to the growth hormone receptor.

### Embodiment 52

An isolated peptide according to any of embodiments 35 to 51, which is an antagonist of the prolactin receptor.

### Embodiment 53

An isolated peptide according to embodiment 52, wherein said antagonism is determined using Assay (II) as described herein.

### Embodiment 54

An isolated peptide according to embodiment 52 or embodiment 53, wherein said antagonism is achieved by introducing one or more mutations into BS2 to prevent or reduce interaction of BS2 with PRL-R.

### Embodiment 55

An isolated peptide according to any of embodiments 52 to 54, wherein at least one or more of said antagonistic mutations are selected from mutations in the amino acid residues corresponding to Gly120 in SEQ ID No. 2.

### Embodiment 56

An isolated peptide according to embodiment 55, wherein at least one or more of said antagonistic mutations are selected from G120R or G120K.

### Embodiment 57

An isolated peptide according to any of embodiments 35 to 51, which is an agonist of the prolactin receptor.

### Embodiment 58

An isolated peptide according to embodiment 57, wherein said peptide binds BS2.

### Embodiment 59

An isolated nucleic acid encoding a peptide according to any of embodiments 1 to 58.

### Embodiment 60

A vector comprising a nucleic acid construct according to embodiment 59.

### Embodiment 61

A host cell comprising a nucleic acid construct of embodiment 59, or a vector of embodiment 60.

### Embodiment 62

An antibody that specifically binds a peptide according to any of embodiments 1 to 58.

### Embodiment 63

An antibody according to embodiment 62, which antibody does not bind to a peptide comprising the amino acid sequence of SEQ ID No. 1.

### Embodiment 64

An antibody according to embodiment 62 or embodiment 63, which antibody does not bind to a peptide comprising the amino acid sequence of SEQ ID No. 2.

### Embodiment 65

A pharmaceutical composition comprising a peptide according to any of embodiments 1 to 58.

### Embodiment 66

A method for treating breast cancer, which method comprising administering a peptide according to any of embodiments 1 to 58 or a formulation according to embodiment 65 to a patient in need thereof.

### Embodiment 67

Use of a peptide according to any of embodiments 1 to 58 for the preparation of a medicament for treatment of breast cancer.

### Embodiment 68

Use of a peptide according to any of embodiments 1 to 58 for generating prolactin antagonists for the treatment of breast and prostate cancers.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

All prolactin molecules used in the examples were expressed in Escherichia coli.

### Example 1—Assay (I)

### Prolactin Receptor Binding Assessed by Surface Plasmon Resonance Measurements

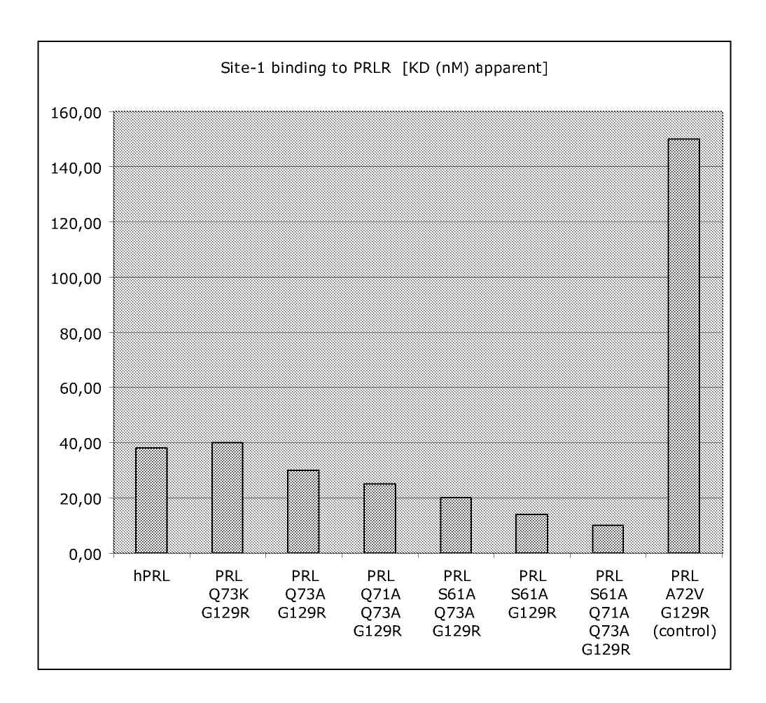

Test compound, in this case the extra-cellular domain of the prolactin receptor (ECD-PRL-R) (25 μg/ml in 10 mM sodium acetate, pH 3.0), was injected into a Biacore 3000 instrument at a flow rate of 5 μl/min and coupled to a CM5 sensor chip by amine coupling chemistry. Prolactin and variants thereof (500 nM in buffer; 20 mM Hepes, pH 7.4, containing 0.1 M NaCl, 2 mM CaCl₂ and 0.005% P20) were then injected over the immobilized receptor for 5 minutes at the same flow rate, followed by a 10-min dissociation period during which buffer was injected, to assess receptor binding affinity. Data evaluation was performed in BiaEvaluation 4.1. Regeneration was accomplished with 4.5 M MgCl₂ between runs. Data for the binding of several PRLP binding compounds are shown in FIG. 3.

### Example 2—Assay (II)

### Phospho-STAT3 ELISA Assay

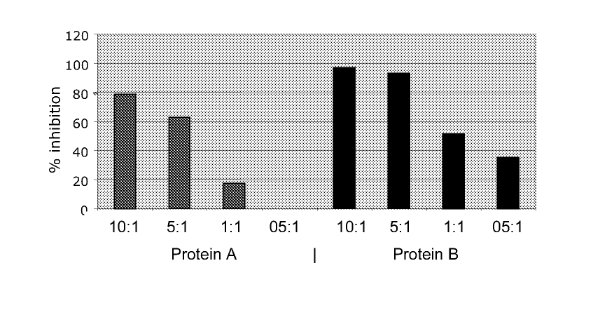

T47D cells grown to approx. 80% confluency were detached with trypsin; cell density was adjusted to 5×10⁵/ml in full growth medium (RPMI, 10% FCS, 2 mM L-glutamin, 0.2 U/ml bovine insulin). 200 μl of this suspension were plated per well of a 96-well plate. The next day, growth medium was replaced with 150 μl starvation medium (growth medium omitting 10% FCS). The cells were starved for 24 hours prior to treatment with PRLR binding compounds. PRL and inhibitors were pre-mixed in starvation medium and 50 μl were added per well to result in 20 nM PRL and varying concentrations of inhibitors indicated in FIG. 4. Protein A is PRL G129R and Protein B is PRL G129R S61A. The cells were incubated for 15 min at 37° C. in a humidified CO₂ incubator. Medium was removed and the cells were washed with ice-cold PBS. Lysis of cells and ELISA were performed according to BioSource STAT-3 [pY705] phospho ELISA manual. The percentage of inhibition of PRL induced STAT3 phosphorylation is shown in FIG. 4 and the apparent IC50 is shown in the Table below.

| | Apparent IC50 (nM) |
|---|---|
| Compound | at 20 nM PRL |
| Protein A: PRL G129R | 69 |
| Protein B: PRL S61A G129R | 18 |

### Example 3—Assay (III)

### Phosphor-STAT5 Reporter Assay.

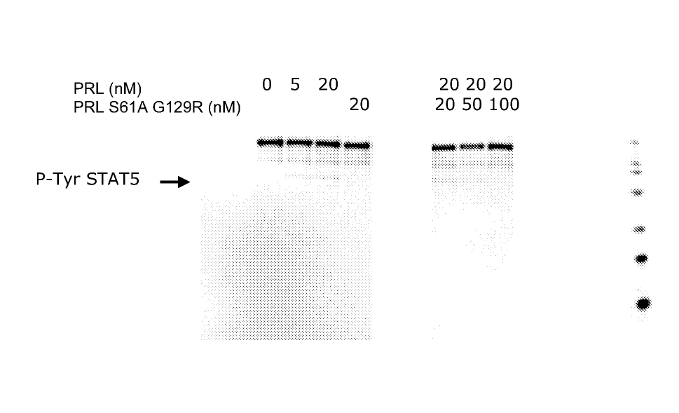

AU 565 cells was cultured for 2 days in 6-well dishes. Cells was starved for 18 hours in growth medium with <1% FCS prior to treatment with PRLR binding compounds. The cells was incubated for 15 min at 37° C. in a humidified CO₂ incubator after addition of varying concentrations of inhibitors as indicated in FIG. 5. Cell lysate was prepared and analyzed for STAT5 tyrosin phosphorylation by western blotting. The results for PRL S61A G129R (labelled 0028) can be seen in FIG. 5.

### Example 4—Assay (IV)

### BaF3 Proliferation Assay for Testing PRLR Antagonists

The BaF/3 cells stably transfected with hPRLR were maintained in the full growth RPMI1640 medium supplemented with 2 mM L-glutamine, 10% FCS and 10 ng/ml wtPRL. Cell were splitted approx. every third day. Prolactin was added upon splitting. Before running the assay, the cells were grown in the medium omitting PRL for 24 hours. The cells were resuspended in fresh medium to 5×10⁵ cells/ml. 100 μl of the cells were fed into wells of a 96-well plate, 50 μl of agonist or wtPRL (1 nM)/antagonists at different concentrations were added to the cells, and the cells were incubated for 68 hours. 50 μl of AlamarBlue (medium: AlamarBlue reagent=7:1) was added to each well, and the cells were then incubated for additional 4 hours. The fluorescence was measured on a BMG LABTECH microplate reader using Ex 544 nm; Em 590 nm.

| Compound | EC50 (nM) | n |
|---|---|---|
| PRL | 0.15 ± 0.08 | 31 |
| PRL in the presence of | 40 ± 18 | 9 |
| 1 μM PRL S61A G129R | | |

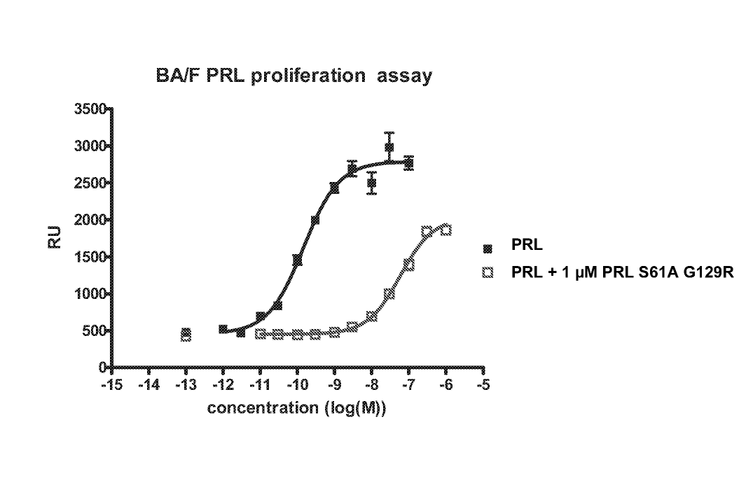

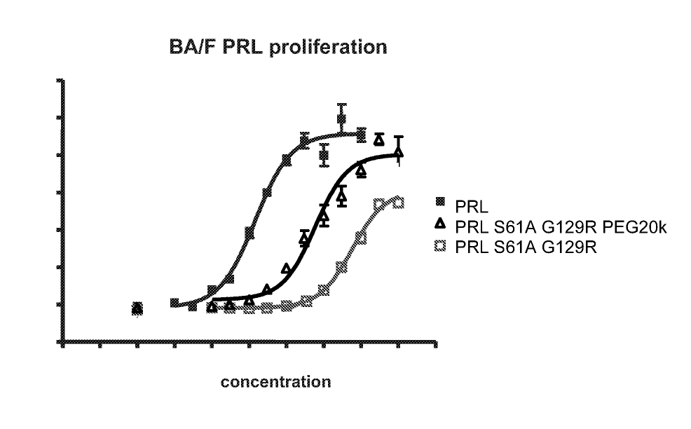

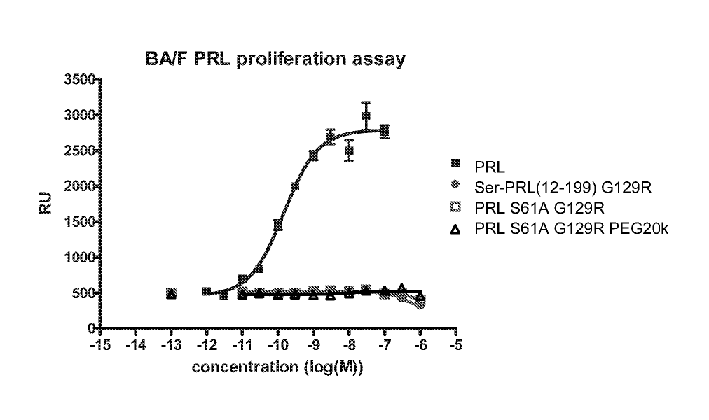

Representative results are also shown in FIGS. 6, 7 and 8.

Preparation of N^{alpha1}-((3-(20 kDa-mPEGyl)propyl)methionyl)PRL S61A G129R

PRL S61A G129R (10 mg, 433 nmol) was dissolved in a mixture of water (0.200 ml) and ethyldiisopropylamine (0.004 ml). A buffer (0.300 ml) consisting of 25 mM MES, which had been adjusted to pH 6.8 by addition of aqueous sodium hydroxide, was added. A 6 M aqueous solution of sucrose (0.80 ml) was added. The mixture was adjusted to pH 6.77 by addition of a 10% solution of acetic acid in water (0.015 ml). A solution of 3-(20 kDa-mPEGyl)-propanal (commercially available at NOF Corporation, nr.: Sunbright ME-200AL, 5 mg, 216 nmol) in a buffer (0.300 ml) consisting of 25 mM MES, which had been adjusted to pH 6.8 by addition of aqueous sodium hydroxide, was added. The pH was adjusted to pH 6.68 by addition of a 10% aqueous solution of acetic acid (0.003 ml). A buffer (0.100 ml), consisting of 25 mM MES, which had been adjusted to pH 6.8 by addition of aqueous sodium hydroxide, was added. The mixture was left at room temperature for 15 min. An 1 M aqueous solution of sodium cyanoborohydride (0.0025 ml) was added. The reaction mixture was gently shaken at 21° C. After 1 h, an 1 M aqueous solution of sodium cyanoborohydride (0.0025 ml) was added. Again after 1 h, an 1 M aqueous solution of sodium cyanoborohydride (0.0025 ml) was added. Again after 1 h, an 1 M aqueous solution of sodium cyanoborohydride (0.0025 ml) was added. The reaction mixture was gently shaken at 21° C. for 16 h. It was diluted with a buffer consisting of 25 mM TRIS, which had been adjusted to pH 8.5 by addition of sodium hydroxide, was added, to obtain a total volume of 4 ml. The reaction mixture was filtered. It was subjected to a gel-chromatography, using a HiPrep Desalting 26/10 column and a buffer, consisting of 25 mM TRIS, which had been adjusted to pH 8.5 by addition of sodium hydroxide. The fractions, containing protein were pooled. They were subjected to an anion-exchange chromatography using a MonoQ 10/100 column and a gradient of 0-75% over 30 CV of a buffer consisting of 25 mM TRIS and 0.2 M sodium chloride, which had been adjusted to pH 8.5 by addition of sodium hydroxide, in a buffer consisting of 25 mM TRIS, which had been adjusted to pH 8.5 by addition of sodium hydroxide. The fractions containing the desired protein were pooled according to their purity estimated by SDS-gel electrophoresis. The pool was divided into two parts. Both parts were subjected to a gel-chromatography, using a HiPrep Desalting 26/10 column and a buffer consisting of 50 mM ammonium hydrogencarbonate. All fractions, containing the desired protein were pooled. The desired product was characterized by SDS-gel, which was stained by PEG-sensitive staining as well as silver stain. Both PEG-stain and silver stain methods show one single compound at a MW in accordance for the expectation of Nalpha1-(3-(20 kDa-mPEGyl)propyl)PRL S61 G129R. The solution was lyophilized. The residue was redissolved in a buffer consisting of 50 mM ammonium hydrogencarbonate and was filtered to obtain a total volume of 1.7 ml. The concentration was determined by spectrometry at 280 nm using a NanoDrop apparatus, employing an extinction coefficient of 8.97. A protein concentration of 0.27 mg/ml was found. Therefore a yield of 0.857 mg of N^{alpha1}-((3-(20 kDa-mPEGyl)propyl)methionyl)PRL S61A G129R (“PRL S61A G129R PEG20k”) was found.

1. An isolated peptide, which peptide is a variant of human prolactin, and which binds to the prolactin receptor, said variant having one or more amino acid mutations in the positions corresponding to positions 61, 71 and 73 of SEQ ID No. 1.

2. An isolated peptide, which peptide is a variant of human prolactin, and which binds to the prolactin receptor, and which peptide comprises the amino acid sequence of SEQ ID No. 1 having one or more amino acid mutations in the positions corresponding to positions 61, 71 and 73 of SEQ ID No. 1.

3. An isolated peptide according to claim 1 having an amino acid mutation in the position corresponding to position 61 of SEQ ID No. 1.

4. An isolated peptide according to claim 1 having an amino acid mutation in the position corresponding to position 71 of SEQ ID No. 1.

5. An isolated peptide according to claim 1 4 having an amino acid mutation in the position corresponding to position 73 of SEQ ID No. 1.

6. An isolated peptide according to claim 1, wherein said peptide has an increased affinity to the prolactin receptor as compared to human prolactin.

7. An isolated peptide according to claim 1, which is an antagonist of the prolactin receptor.

8. An isolated peptide, which peptide is a variant of human growth hormone, and which binds to the growth hormone receptor, said variant having one or more amino acid mutations in the positions corresponding to positions 61, 71 and 73 of SEQ ID No. 1.

9. An isolated peptide according to claim 8, wherein the peptide has an increased binding to the prolactin receptor through binding site 1 as compared to human prolactin.

10. An isolated nucleic acid encoding a peptide according to claim 1.

11. A vector comprising a nucleic acid construct according to claim 10.

12. A host cell comprising a nucleic acid construct of claim 10.

13. An antibody that specifically binds a peptide according to claim 1.

14. An antibody according to claim 13, which antibody does not bind to a peptide comprising the amino acid sequence of SEQ ID No. 1.

15. An antibody according to claim 13, which antibody does not bind to a peptide comprising the amino acid sequence of SEQ ID No. 2.

16. A pharmaceutical composition comprising a peptide according to claim 1.

17. A method for treating breast cancer, which method comprising administering a peptide according to claim 1 to a patient in need thereof.

18-19. (canceled)

20. An isolated nucleic acid encoding a peptide according to claim 8.
